(19) United States
(12) Patent Application Publication
(10) Pub. No.: US 2004/0220094 A1
(43) Pub. Date: Nov. 4, 2004

(54) Inverse agonist and agonist peptides that stimulate/inhibit hair growth

(76) Inventor: Keith K. Skinner, Honolulu, HI (US)

Correspondence Address: MORRISON & FOERSTER LLP, 3811 VALLEY CENTRE DRIVE, SUITE 500, SAN DIEGO, CA, 92130-2332, US

(21) Appl. No.: 10/428,377

(22) Filed: May 1, 2003

(51) Int. Cl. A61K 38/10; C07K 14/47

(52) U.S. Cl. 514/12; 530/324

(57) ABSTRACT

The invention provides peptides and compositions comprising them for the treatment of hair loss and excess hair as well as psoriasis. Methods of preparing the peptides as well as methods for their use are also provided as well as methods of assaying them for inverse agonist and agonist activities. The compounds of the present invention may be topically administered to areas of skin requiring such treatment.

### TECHNICAL FIELD

The invention relates to peptides that stimulate hair growth, and peptides that inhibit hair growth on skin, as well as compositions and methods of using said peptides. The peptides are inverse agonists and agonists of two human peptide hormones in their action upon a receptor. Inverse agonist peptides of the invention may be used to treat alopecia while agonist peptides may be used to inhibit hair growth at desired locations on skin. The agonist peptides may also be used to inhibit psoriasis. Pharmaceutical formulations of the peptides are provided for their use. The invention also relates to the methods of preparing the peptides of the invention as well as to methods of screening peptides for inverse agonist and agonist activities.

### BACKGROUND ART

Human parathyroid hormone-related protein (PTHrP) is a 141 amino acid peptide. PTHrP is expressed in various tissues (including the skin & hair follicle) where it acts as an endocrine/paracrine factor involved in cellular growth. (Juppner et al, 1991; Urena et al, 1993). PTHrP is strongly expressed in the epidermis and has been implicated in the regulation of growth and differentiation of keratinocytes and hair follicle cells. PTHrP has had an unidentified role in medicine since 1930 (P R Health Sci J Mar. 16, 1997;(1):15-22).

Human parathyroid hormone (PTH) has a length of 84 amino acids and is also expressed in various tissues (including the skin) where it also acts as an endocrine/paracrine factor involved in cellular growth. (Juppner et al, 1991; Urena et al, 1993).

Although the full length versions of peptide hormones PTHrP and PTH are much longer, amino acids 1 to 34, inclusive, from the N-terminals of each of human PTHrP and human PTH elicit the full spectrum of skin and hair follicle-relevant activities characteristic of the intact (full length) hormones. (Biochemistry. Jun. 25, 2002;41(25):8162-75, and Mol Cell Endocrinol Mar. 28, 2002;189(1-2):37-49).

PTHrP and PTH exert their activity via the Type I PTH/PTHrP receptor (PTH1R receptor). The PTH1R receptor is substantially alpha-helical in nature; is a specific G protein-coupled, seven-transmembrane helix-containing receptor; has an apparent molecular weight of approximately 85000; and contains four putative N-glycosylation sites. Skin fibroblasts possess this PTH/PTHrP (PTH1R) receptor and are target cells for PTH and PTHrP whereas keratinocytes do not have the receptor and are unresponsive to it. The receptor thus permits the targeting of fibroblasts and inner root sheath cells for skin and hair follicle modulation. (J. Invest Dermatol July 1995;105(1):133-7).

U.S. Pat. No. 6,495,662 describes bioactive peptides and peptide derivatives of PTHrP and PTH in relation to bone growth. U.S. Pat. Nos. 5,527,772 and 5,840,690 as well as U.S. Pat. Nos. 5,958,384 and 5,744,128 describe the use of some PTH/PTHrP peptides for stimulating skin and hair growth.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

Disclosure of the Invention

The invention provides novel peptides which are inverse agonists of PTHrP and PTH activity on the PTH1R receptor. It is believed that this is the first instance of inverse agonists of this receptor as well as the beneficial use thereof to stimulate hair growth. Inverse agonist peptides are advantageously used to stimulate hair growth in alopecia of various origins as well as to treat any skin condition requiring inverse agonist or antagonist activity on the PTH1R receptor. The invention also provides novel peptides which are agonists of PTHrP and PTH activity on the PTH1R receptor. These peptides are advantageously used to inhibit hair growth and psoriasis as well as to treat any skin condition requiring such agonist activity.

The peptides of the invention have been identified to be a range of agonist and inverse agonist activities. This classification of peptides includes the existence of full and partial agonists, which refer to agonist compounds which produce 100% or less than 100% of the activity of a receptor for said compounds. This is in contrast to an antagonist, which competes against an agonist to prevent agonist mediated activity. An inverse agonist reverses the agonist mediated activity. Inverse agonists of the invention compete against regular agonist and antagonist compounds for binding. But by the design of inverse agonists that dissociate slowly, binding by regular agonist and antagonist compounds is effectively reduced. Inverse agonists that reverse PTH1R receptor activities such as increases in intracellular calcium and cAMP levels are the preferred compounds for use in the present invention to stimulate hair growth. Particularly preferred peptides are those that decrease intracellular calcium and cAMP levels via the PTH1R receptor. Assays that detect intracellular calcium and cAMP levels are also provided by the invention to determine the level of agonist and inverse agonist activity by the peptides of the invention.

The invention is based in part on the recognition that peptides smaller than full length PTHrP and PTH, as well as PTHrP 7-34, are advantageously delivered to hair follicles and dispersed through the skin via topical application. The invention is also based in part upon the recognition that the antagonist activity of PTHrP 7-34 peptide is not potent enough to grow hair on humans. The problem with PTHrP 7-34 is that it dissociates very rapidly from the human PTH1R receptor (t_{1/2} values of about 10 seconds (Hoare S R, Usdin T B. Tuberoinfundibular peptide (7-39), or TIP(7-39), a novel, selective, high-affinity antagonist for the parathyroid hormone-1 receptor with no detectable agonist activity. J Pharmacol Exp Ther 2000;295:761-70.).

In one aspect, the invention provides synthetically, or recombinantly, produced inverse agonist peptides that stimulate hair growth. These peptides include derivatives of PTHrP 7-34 and derivatives of amino acids 7-34 of PTH. Without being bound by theory, and offered to improve the understanding of the invention, the peptides bind the PTH1R receptor via a ‘two-site’ mechanism in which the C-terminal portions of the peptides bind the extracellular N-terminal domain of the receptor (N-interaction), and the N-terminal portions of the peptides bind to the juxtamembrane receptor domain (J-interaction). The (N-interaction) provides most of the PTH1R receptor binding energy for the peptides while the (J-interaction) stimulates G-protein activation. For the PTH-PTH1R receptor interaction, the efficacy-generating component of the (J-interaction) is independent of the N-domain of the receptor and C-terminal portion of the peptide.

The invention provides derivatives of amino acids 7-34 of human and bovine, PTH and PTHrP as follows. The sequence of human PTH 7-34 is

Leu-Met-His-Asn-Leu-Gly-Lys-His-Leu-Asn-Ser-Met-
Glu-Arg-Val-Glu-Trp-Leu-Arg-Lys-Lys-Leu-Gln-Asp-
Val-His-Asn-Phe.

The sequence of human PTHrP 7-34 is

Leu-Leu-His-Asp-Lys-Gly-Lys-Ser-Ile-Gln-Asp-Leu-
Arg-Arg-Arg-Phe-Phe-Leu-His-His-Leu-Ile-Ala-Glu-
Ile-His-Thr-Ala.

The sequence of bovine PTH 7-34 and PTHrP 7-34 is

Phe-Met-His-Asn-Leu-Gly-Lys-His-Leu-Ser-Ser-Met-
Glu-Arg-Val-Glu-Trp-Leu-Arg-Lys-Lys-Leu-Gln-Asp-
Val-His-Asn-Phe.

In one embodiment, the invention provides a peptide represented by Formula 1:

| | |
|---|---|
| X1-X2-His-Asn-X5-X6-X7-X8-X9-X10-X11-X12-X13-Arg- | tl,32 |
| X15-X16-X17-Leu-X19-X20-X21-X22-X23-X24-X25-His- | |
| X27-X28 | |

wherein X1 is independently leucine or phenylalanine; X2 is independently methionine, leucine, or norleucine; X5 is independently leucine or lysine; X6 is independently glycine, D-tryptophan, D-α-naphthylalanine (D-α-Nal) or D-β-Nal; X7 is lysine, optionally substituted at its epsilon amino group; X8 is independently serine or histidine; X9 is independently leucine or isoleucine; X10 is independently asparagine, glutamine, serine or alanine; X11 is independently serine or aspartic acid; X12 is independently methionine or leucine, norleucine or valine; X13 is independently glutamic acid or arginine; X15 is independently valine, arginine or methionine; X16 is independently glutamic acid, phenylalanine or glutamine; X17 is independently tryptophan or phenylalanine; X19 is independently arginine or histidine; X20 is independently lysine or histidine; X21 is independently lysine or leucine; X22 is independently leucine or isoleucine; X23 is independently glutamine or alanine; X24 is independently aspartic acid or glutamic acid; X25 is independently valine or isoleucine; X27 is independently asparagine or threonine; and X28 is independently phenylalanine, tyrosine, or alanine.

In some embodiments of the invention, the peptide is preferably not human or bovine PTHrP7-34.

Optional substituents at the epsilon amino group of the X7 lysine are selected from hydrophobic moieties, such as phenylpropanoyl, and biotin containing moieties, such as biotin or biotinyl-beta-Ala. Incorporation of orthogonally protected N alpha-Boc-Lys(N epsilon-Fmoc) at a selected position in the sequence, such as the X7 lysine residue, followed by selective side-chain deprotection and biotinylation of the epsilon-amino group, permits modification of the specific lysine only. Alternatively, the X7 Lys residue may be substituted by a hydrophobic uncharged residue such as Phe, Ile, Leu, Met, Val, Trp, and Tyr.

The glycine residue at position X6 (position 12 of PTHrP 7-34) as shown above participates in a β-turn into the peptide and thus may be substituted in any PTHrP 7-34 derived peptide of the invention by D-tryptophan, D-α-naphthylalanine (D-α-Nal) or D-β-Nal. Examples of such substituted peptides increased potency of inducing hair growth by 10 fold over unsubstituted peptides. The invention is based in part on the discovery that a turn of approximately 15 degrees in the peptide backbone at positions X5 through X7 (positions 11 through 13 of PTH or PTHrP) increases the potency of the peptides of the invention. This turn is thus preferably present in the peptides disclosed herein, including those of the formulas below. The potency of peptides having this turn is further increased by positions X1 to X5 and positions X14 to X28 being in alpha helices or alpha helical structures.

The methionine residues at positions X2 and X12 (positions 8 and 18 of PTHrP 7-34) as shown above are independently leucine or norleucine (Nle) in any PTHrP 7-34 derived peptide of the invention.

Particularly preferred embodiments of this aspect of the invention are peptides of the sequence

Leu-Leu-His-Asn-Leu-Gly-Lys-Ser-Ile-Gln-Asp-Leu-
Arg-Arg-Arg-Phe-Phe-Leu-His-His-Leu-Ile-Ala-Glu-
Ile-His-Thr-Ala-NH₂
(which is 23 fold more potent than PTHrP 7-34);
Leu-Leu-His-Asn-Leu-D-Trp-Lys-Ser-Ile-Gln-Asp-Leu-
Arg-Arg-Arg-Phe-Phe-Leu-His-His-Leu-Ile-Ala-Glu-
Ile-His-Thr-Ala-NH₂;
(which is 26 fold more potent than PTHrP 7-34);
Leu-Leu-His-Asp-Leu-D-Trp-Lys-Ser-Ile-Gln-Asp-Leu-
Arg-Arg-Arg-Phe-Phe-Leu-His-His-Leu-Ile-Ala-Glu-
Ile-His-Thr-Ala;
Phe-Met-His-Asn-Leu-Gly-Lys-His-Leu-Ser-Ser-Met-
Glu-Arg-Val-Glu-Trp-Leu-Arg-Lys-Lys-Leu-Gln-Asp-
Val-His-Asn-Tyr;
Phe-Nle-His-Asn-Leu-Gly-Lys-_{(epsilon-3-phenyl-}
_{propanoyl)}-His-Leu-Ser-Ser-Nle-Glu-Arg-Val-Glu-
Trp-Leu-Arg-Lys-Lys-Leu-Gln-Asp-Val-His-Asn-Tyr
(K_{binding} = 4 and 9 nM, Kᵢ = 73 and 3.5 nM in
kidney- and bon-based assays, respectively); and
Leu-Leu-His-Asp-Leu-D-Trp-Lys-_{(N epsilon-}
_{(biotinyl-beta-Ala))}-Ser-Ile-Gln-Asp-Leu-Arg-Arg-
Arg-Phe-Phe-Leu-His-His-Leu-Ile-Ala-Glu-Ile-His-
Thr-Ala

In another embodiment, the invention provides a cyclized peptide represented by Formula 2:

X1-X2-His-Asn-X5-X6-Lys-X8-X9-X10-Asp-X12-X13-Arg-
X15-X16-X17-Leu-X19-X20-X21-X22-X23-X24-X25-His-
X27-X28

wherein the lysine between positions X6 and X8 (corresponding to position 13 in PTHrP 7-34) and the aspartic acid between positions X10 and X12 (corresponding to position 17 in PTHrP 7-34) are linked by a covalent bond between the epsilon-amino of the lysine and the beta-carboxyl of the aspartic acid to yield a 20-membered ring lactam;

wherein X1 is independently leucine or phenylalanine; X2 is independently methionine, leucine, or norleucine; X5 is independently leucine or lysine; X6 is independently glycine, D-tryptophan, D-α-naphthylalanine (D-α-Nal) or D-β-Nal; X8 is independently serine or histidine; X9 is independently leucine or isoleucine; X10 is independently asparagine, glutamine, serine or alanine; X12 is independently methionine or leucine, norleucine or valine; X13 is independently glutamic acid or arginine; X15 is independently valine, arginine or methionine; X16 is independently glutamic acid, phenylalanine or glutamine; X17 is independently tryptophan or phenylalanine; X19 is independently arginine or histidine; X20 is independently lysine or histidine; X21 is independently lysine or leucine; X22 is independently leucine or isoleucine; X23 is independently glutamine or alanine; X24 is independently aspartic acid or glutamic acid; X25 is independently valine or isoleucine; X27 is independently asparagine or threonine; and X28 is independently phenylalanine, tyrosine, or alanine-NH₂.

The cyclized peptide of Formula 2 having the sequence of human PTHrP 7-34, denoted [Lys13,Asp17]PTHrP(7-34)NH₂, was 5-10-fold more potent than the linear parent peptide in receptor binding studies (K_{binding}=15 and 18 nM) in contrast, a linear analogue in which charges in positions 13 and 17 were eliminated and other stereoisomers of the above-mentioned lactam in which either Lys13 and/or Asp17 were replaced by the corresponding D-amino acids were much less potent with regard to inverse agonist bioactivity than the parent peptide.

One cyclized peptide of this formula has the sequence

Leu-Leu-His-Asp-Leu-D-Trp-Lys_{(cyclization)}-Ser-
Ile-Gln-Asp_{(cyclization)}-Leu-Arg-Arg-Arg-Phe-Phe-
Leu-His-His-Leu-Ile-Ala-Glu-Ile-His-Thr-Ala-NH₂.

This peptide showed increased helicity at positions X1 to X5, and at least positions X14 to X28 in the presence of a cationic surfactant as well as increased potency.

Without being bound by theory, and provided to improve understanding of the invention, hydrophobic amino acids at positions corresponding to positions 11 and/or 12 of the cyclized PTHrP above contribute to an increase in binding affinity by increasing hydrophobic interactions which stabilize receptor-peptide complexes. Structural rigidification provided by cyclization increases the alpha-helical content, which is important for attaining a peptide conformation recognized by the receptor.

In another embodiment of cyclized peptides, the invention provides peptides represented by Formula 3:

X1-X2-His-Asn-X5-X6-X7-X8-X9-X10-X11-X12-X13-Arg-
X15-Glu-X17-Leu-X19-Lys-X21-X22-X23-X24-X25-His-
X27-X28

wherein the glutamic acid between positions X15 and X17 (corresponding to position 22 in PTHrP 7-34) and the lysine between positions X19 and X21 (corresponding to position 22 in PTHrP 7-34) are linked by a covalent bond between the gamma-carboxyl of the aspartic acid and the epsilon-amino of the lysine to yield a lactam ring;

wherein X1 is independently leucine or phenylalanine; X2 is independently methionine, leucine, or norleucine; X5 is independently leucine or lysine; X6 is independently glycine, D-tryptophan, D-α-naphthylalanine (D-α-Nal) or D-β-Nal; X7 is independently lysine, optionally substituted at its epsilon amino group; X8 is independently serine or histidine; X9 is independently leucine or isoleucine; X10 is independently asparagine, glutamine, serine or alanine; X11 is independently serine or aspartic acid; X12 is independently methionine or leucine, norleucine or valine; X13 is independently glutamic acid or arginine; X15 is independently valine, arginine or methionine; X17 is independently tryptophan or phenylalanine; X19 is independently arginine or histidine; X21 is independently lysine or leucine; X22 is independently leucine or isoleucine; X23 is independently glutamine or alanine; X24 is independently aspartic acid or glutamic acid; X25 is independently valine or isoleucine; X27 is independently asparagine or threonine; and X28 is independently phenylalanine, tyrosine, or alanine-NH2.

One cyclized peptide of this formula has the sequence

Leu-Met-His-Asn-Leu-Gly-Lys-His-Leu-Asn-Ser-Met-
Glu-Arg-Val-Glu_{(cyclization)}-Trp-Leu-Arg-
Lys_{(cyclization)}-Lys-Leu-Gln-Asp-Val-His-Asn-Phe.

Cyclized peptides according to Formula 3 have enhanced structural stability within the amphiphilic helix critical PTH1R receptor binding region of positions 21-31 of PTHrP 7-34 (positions X15 to X25 of Formula 3). Further enhanced structural stability of the peptides of Formula 3 may be provided to this region by other side-chain to side-chain cyclization between amino acids that are four residues apart (from I to I+4) such as the following peptide

Leu-Leu-His-Asp-Lys-Gly-Lys-Ser-Ile-Gln-Asp-Leu-
Arg-Arg-Arg-Phe-Phe-Leu-His-His_{(cyclization)}-Leu-
Ile-Ala-Glu_{(cyclization)}-Ile-His-Thr-Ala-NH₂

Doubly cyclized peptides according to Formulas 2 and 3 as described above are also provided by the present invention. Preferably, such doubly cyclized peptides are further substituted at positions X1 to X5 and X21 to X28 (or X25 to X28 in the case of the His to Glu cyclization provided above) with amino acids that maintain an alpha helical structure at those positions.

In another aspect of the invention, preferred PTHrP derived peptides according to the above, positions 12 and 13 of PTHrP 7-34 (corresponding to positions X5 and X6 of the above formulas), as well as position 19 of PTHrP 7-34 (corresponding to position X13 of the above formulas), participate in “hinge” regions of the peptides. As noted above, positions X5 through X7 form a turn of about 15 degrees, which, combined with a “hinge” or bend in the backbone at an arginine at position X13, form a structural backbone that is preferred for the practice of the invention. This backbone is represented by the following Formula 4:

X1-X2-X3-X4-X5-X6-Lys-X8-X9-X10-X11-X12-Arg-X14-
X15-X16-X17-X18-X19-X20-X21-X22-X23-X24-X25-X26-
X27-X28

wherein X5, X6 and the Lys residue at X7 form a turn of about 15 degrees and positions X1-X4 as well as X14 to X18 form alpha helical structures; and

X8 to X12 is His/Ser-Leu/Ile-Ser/Asn/Gln/Ala-Ser/Asp-Met/Leu/Nle/Val. The Lys at position X7 is optionally substituted as described above.

Preferably, X1-X4 as well as X14 to X18 are the amino acid sequences of a naturally occurring PTH or PTHrP that has been substituted at one or more residues with an amino acid that forms or stabilizes alpha helical structures. Such amino acid substitutions are described below and preferably do not result in the retention of a naturally occurring sequence, such as conversion of a bovine sequence into a human sequence. More preferred are peptides where X14 is Arg; X16 is Glu; X18 is Leu; X20 is Lys; X26 is His.

The peptides of Formula 4 may optionally comprise single or double cyclization as present above. Particularly preferred is cyclization between Lys at X7 and Asp at X11 as described above, which also permits other amino acid substitutions at positions X8 to X10. A second cyclization between a Glu residue at X14 and a Lys residue at X18 as described above is also preferred in the practice of the invention; this cyclization also permits other amino acid substitutions at positions X15 to X17. Alternatively, the second cyclization can be between a His residue at X26 and a Glu residue at X30 as described above; this cyclization also permits other amino acid substitutions at positions X27 to X29. Doubly cyclized peptides with alpha helical structures at positions X1 to X4 and at least X31 to X34 are also provided by the present invention.

In another aspect of the invention, peptides derived from amino acids 7-39 of tuberoinfundibular peptide (TIP) are provided. The sequence of human/bovine TIP 7-39 is

Asp-Ala-Ala-Phe-Arg-Glu-Arg-Ala-Arg-Leu-Leu-Ala-
Ala-Leu-Glu-Arg-Arg-His-Trp-Leu-Asn-Ser-Tyr-Met-
His-Lys-Leu-Leu-Val-Leu-Asp-Ala-Pro.

The mouse sequence is

Asp-Ala-Ala-Phe-Arg-Glu-Arg-Ala-Arg-Leu-Leu-Ala-
Ala-Leu-Glu-Arg-Arg-Arg-Trp-Leu-Asn-Ser-Tyr-Met-
Gln-Lys-Leu-Leu-Leu-Leu-Asp-Ala-Pro

In one embodiment, the invention provides a peptide represented by Formula 5:

Asp-Ala-Ala-Phe-Arg-Glu-Arg-Ala-Arg-Leu-Leu-Ala-
Ala-Leu-Glu-Arg-Arg-X18-Trp-Leu-X21-Ser-Tyr-X24-
X25-Lys-Leu-Leu-X29-Leu-Asp-Ala-Pro

wherein X18 is independently histidine or arginine; X21 is independently aspartic acid or asparagine; X24 is independently methionine or norleucine; X25 is independently glutamine or histidine; and X29 is independently leucine or valine.

In one embodiment of this aspect, a potent peptide inverse agonist of PTH1R receptor has the sequence

Asp-Ala-Ala-Phe-Arg-Glu-Arg-Ala-Arg-Leu-Leu-Ala-
Ala-Leu-Glu-Arg-Arg-Arg-Trp-Leu-Asp-Ser-Tyr-Met-
Gln-Lys-Leu-Leu-Leu-Leu-Asp-Ala-Pro;
Asp-Ala-Ala-Phe-Arg-Glu-Arg-Ala-Arg-Leu-Leu-Ala-
Ala-Leu-Glu-Arg-Arg-His-Trp-Leu-Asn-Ser-Tyr-Nle-
His-Lys-Leu-Leu-Val-Leu-Asp-Ala-Pro;
Asp-Ala-Ala-Phe-Arg-Glu-Arg-Ala-Arg-Leu-Leu-Ala-
Ala-Leu-Glu-Arg-Arg-His-Trp-Leu-Asn-Ser-Tyr-Met-
His-Lys-Leu-Leu-Val-Leu-Asp-Ala-Pro;
Asp-Ala-Ala-Phe-Arg-Glu-Arg-Ala-Arg-Leu-Leu-Ala-
Ala-Leu-Glu-Arg-Arg-Arg-Trp-Leu-Asp-Ser-Tyr-Met-
Gln-Lys-Leu-Leu-Leu-Leu-Asp-Ala-Pro; and
Asp-Ala-Ala-Phe-Arg-Glu-Arg-Ala-Arg-Leu-Leu-Ala-
Ala-Leu-Glu-Arg-Arg-Arg-Trp-Leu-Asp-Ser-Tyr-Nle-
Gln-Lys-Leu-Leu-Leu-Leu-Asp-Ala-Pro.

The above peptides of the invention have half-life dissociation times from the PTH1R receptor in the range of minutes to over one hour. In some embodiments of the invention, peptides of Formula 5 are not those of human, bovine, or mouse TIP 7-39.

In a further aspect of the invention, peptide agonists of the PTH1R receptor are provided. In addition to inhibiting hair growth, agonists of the invention may also be used to inhibit psoriasis. These peptides include derivatives of amino acids 1-14 of human PTH (MW 4117.77). The amino acid sequence of human PTH is

Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe.

In one embodiment of this aspect, the invention provides a peptide represented by Formula 6:

X1-Val-X3-Glu-Ile-Gln-Leu-X8-His-X10-X11

wherein X1 and X3 are independently serine, alanine, or a-aminoisobutyric acid (Aib); X8 is independently methionine or norleucine; X10 is independently asparagine, alanine, glutamine, or histidine; and X11 is independently leucine, arginine, or homoarginine (Har); and wherein when X10 is histidine, the peptide is bound to divalent zinc salts (e.g. Zn(II)⁺⁺ ions). Aib is a helix-promoting amino acid. This aspect of the invention is based in part on the unexpected discovery that amino acid residues 7 to 11 (positions X7 to X11 of Formula 6) are beneficially present in peptide agonists of the PTH1R receptor. This is in contrast to other work indicating that residues 7 to 11 are part of active peptide antagonists of the receptor. In some embodiments of the invention, the peptides of Formula 6 do not have the sequence of residues 1-11 of human PTH.

The peptide of Formula 6 may also comprise three additional amino acids at its carboxyl terminus to result in a peptide having the formula

X1-Val-X3-Glu-Ile-Gln-Leu-X8-His-X10-X11-X12-Lys-X14

wherein X1, X3, X8, X10, and X11 are as defined above while X12 is independently glycine or alanine; and X14 is independently histidine or tryptophan. In some embodiments of the invention, the peptide does not have the sequence of residues 1-14 of human PTH.

Exemplary embodiments of Formula 6 peptides have the following sequences:

Ser-Val-Ser-Glu-Ile-Gln-Leu-Met-His-Asn-Leu;
Ser-Val-Ser-Glu-Ile-Gln-Leu-Met-His-His-Leu;
Ser-Val-Ala-Glu-Ile-Gln-Leu-Met-His-Gln-Har;
Aib-Val-Aib-Glu-Ile-Gln-Leu-Met-His-Gln-Har-
Ala-Lys-Trp;
Ser-Val-Ser-Glu-Ile-Gln-Leu-Met-His-His-Leu-
Gly-Lys-His;
Ser-Val-Ala-Glu-Ile-Gln-Leu-Met-His-Gln-Har-
Ala-Lys-Trp;
Ser-Val-Ala-Glu-Ile-Gln-Leu-Met-His-Ala-Arg-
Ala-Lys-Trp; and
Ser-Val-Ala-Glu-Ile-Gln-Leu-Met-His-His-Arg-
Ala-Lys-Trp.

In another embodiment of this aspect of the invention, full length analogues of human PTH are provided according to Formula 7:

X1-Val-X3-Glu-Ile-Gln-X7-X8-His-X10-X11-X12-Lys-
X14-X15-X16-X17-X18-X19-Arg-X21-X22-X23-Leu-X25-
X26-X27-X28-X29-X30-X31-His-X33-X34

wherein X1 and X3 are independently serine or alanine; X7 is independently leucine or phenylalanine; X8 is independently methionine, leucine, or norleucine; X10 is independently asparagine, glutamine, aspartic acid, or histidine; X11 is independently leucine, lysine, or homoarginine; X12 is independently glycine or alanine; X14 is independently histidine, serine, or tryptophan; X15 is independently leucine or isoleucine; X16 is independently asparagine, alanine, serine or glutamine; X17 is independently serine or aspartic acid; X18 is methionine, norleucine, valine, or leucine; X19 is glutamic acid or arginine; X21 is independently valine, methionine, norleucine, or arginine; X22 is independently glutamic acid, glutamine, isoleucine, or phenylalanine; X23 is independently tryptophan or phenylalanine; X25 is independently arginine, glutamine, or histidine; X26 is independently lysine, asparagine, or histidine; X27 is independently lysine or leucine; X28 is independently leucine or isoleucine; X29 is independently glutamine, glutamic acid, or alanine; X30 is independently aspartic acid, glycine, or glutamic acid; X31 is independently valine or isoleucine; X33 is independently asparagine or threonine; and X34 is independently phenylalanine, tyrosine, or alanine; and wherein said peptide is not human PTH.

Exemplary embodiments of Formula 7 peptides have the following sequences:

Ala-Val-Ser-Glu-Ile-Gln-Phe-Nle-His-Asn-Leu-Gly-
Lys-His-Leu-Ser-Ser-Nle-Glu-Arg-Val-Glu-Trp-Leu-
Arg-Lys-Lys-Leu-Gln-Asp-Val-His-Asn-Tyr;
Ala-Val-Ser-Glu-Ile-Gln-Leu-Nle-His-Asn-Leu-Gly-
Lys-His-Leu-Ala-Ser-Val-Glu-Arg-Nle-Gln-Trp-Leu-
Arg-Lys-Lys-Leu-Gln-Asp-Val-His-Asn-Tyr;
Ala-Val-Ser-Glu-Ile-Gln-Phe-Met-His-Asn-Leu-Gly-
Lys-His-Leu-Ser-Ser-Met-Glu-Arg-Val-Glu-Trp-Leu-
Arg-Lys-Lys-Leu-Gln-Asp-Val-His-Asn-Tyr;
Ala-Val-Ser-Glu-Ile-Gln-Phe-Nle-His-Asn-Leu-Gly-
Lys-His-Leu-Ser-Ser-Nle-Glu-Arg-Val-Glu-Trp-Leu-
Arg-Lys-Lys-Leu-Gln-Asp-Val-His-Asn-Tyr;
Ser-Val-Ser-Glu-Ile-Gln-Leu-Nle-His-Asn-Leu-Gly-
Lys-His-Leu-Asn-Ser-Nle-Glu-Arg-Val-Glu-Trp-Leu-
Arg-Lys-Lys-Leu-Gln-Asp-Val-His-Asn-Tyr;
Ala-Val-Ser-Glu-Ile-Gln-Leu-Met-His-Asn-Leu-Gly-
Lys-His-Leu-Ala-Ser-Val-Glu-Arg-Nle-Gln-Trp-Leu-
Arg-Lys-Lys-Leu-Gln-Asp-Val-His-Asn-Phe; and
Ala-Val-Ser-Glu-His-Gln-Leu-Leu-His-Asp-Lys-Gly-
Lys-Ser-Ile-Gln-Asp-Leu-Arg-Arg-Arg-Phe-Phe-Leu-
His-His-Leu-Ile-Ala-Glu-Ile-His-Thr-Tyr.

A further embodiment is the following 36 amino acid peptide:

Ala-Val-Ser-Glu-His-Gln-Leu-Leu-His-Asp-Lys-Gly-
Lys-Ser-Ile-Gln-Asp-Leu-Arg-Arg-Arg-Ile-Phe-Leu-
Gln-Asn-Leu-Ile-Glu-Gly-Val-Asn-Thr-Ala-Glu-Tyr.

Without being bound by theory, and offered to improve the understanding of the invention, position 19 (Glu) in the full length human PTH is preferably alpha-helical for optimal interaction with the juxtamembrane portion of the receptor.

The invention also provides methods for the preparation of the peptides of the invention. As would be clear to the skilled person, peptides of the invention may be prepared synthetically (exemplified by solid or liquid phase synthesis as a non-limiting example) or recombinantly where only naturally occurring amino acid residues are present in the peptide. Peptides with non-naturally occurring amino acid residues (such as the D form of amino acid residues, α-aminoisobutyric acid or the D-α- and D-β-forms of naphthylalanine) are preferably prepared synthetically. When prepared recombinantly in cells, the peptides of the invention are preferably expressed in a form that is secreted into the growth medium (or extracellular space) in which the cells are cultured. In some embodiments, such secretion may be effected by the use of a cleavable pro-peptide which is expressed as a fusion peptide with a peptide of the invention. The pro-peptide may be cleaved in combination with the secretion of said peptide into the medium.

The peptides of the invention may also optionally contain additional atoms, moieties, or amino acid residues, particularly at the amino or carboxyl terminals of the peptide. A non-limiting example includes the presence of an amino group (—NH2) at the carboxyl terminal of a peptide of the invention. Such embodiments of the invention include, but are not limited to, a fusion protein comprising a peptide of the invention wherein the fusion protein may be recombinantly expressed and isolated followed by specific proteolytic cleavage to release the peptide of the invention. Another non-limiting example are peptides acylated at the N— or C— terminus with a moiety of at least about 5 to about 22 carbon atoms, such as to result in palmitoylation, myristoylation, and farnesylation of the peptides. Other modifications include, but are not limited to, acetylation, amidation, phosphorylation, and glycosylation.

The present invention also provides compositions comprising the peptides disclosed herein and at least one pharmaceutically acceptable excipient. Such compositions may be used in methods of inverse agonizing or agonizing the PTH1R receptor as well as in methods of stimulating or inhibiting hair growth. The compositions of the invention may optionally comprise other agents effective in the intended use of the composition. As non-limiting examples, compositions comprising an inverse agonist peptide of the invention may further comprise another agent which stimulates hair growth; compositions comprising an agonist peptide of the invention may further comprise another agent which inhibits hair growth or psoriasis.

An increase or decrease in hair growth preferably relates to terminal hairs and/or vellus hairs as well as being defined in terms of hair count. Terminal hairs are long, pigmented hairs that are produced by follicles with sebaceous (oil) glands. They are found on the scalp, beard, armpits and pubic areas and are in contrast to vellus hairs, which are short hairs, often only a centimetre or two long, that contain little or no pigment. Terminal hairs also differ from Lanugo hair, which develops on an unborn baby.

Alternatively, a peptide of the invention is labeled such that it may be visible, made visible, or otherwise readily detected. Such labeled peptides may be used to localize PTH1R receptors or cells comprising them.

Furthermore, the present invention also provides methods of determining the level of inverse agonist or agonist activity by the peptides of the invention, as well as for the abilities to stimulate or inhibit hair growth, or inverse agonizing or agonizing the PTH1R receptor.

Modes of Carrying Out the Invention

As noted herein, the invention includes the substitution of amino acid residues by other amino acids with the ability to stabilize or form alpha helices or alpha helical structures. While means for the selection and substitution of alpha helix forming amino acids are well know in the art, the selection may be viewed as substituting one amino acid residue by another that is more likely to stabilize or form an alpha helix. This may be accomplished by comparing amino acid residues on a relative scale of alpha helix occurrence. The following table lists the 20 naturally occurring amino acids in order of their relative frequency in alpha helices (T. E. Creighton. Proteins: Structures and Molecular Properties, W. H. Freeman, 1983, pg. 235).

| | |
|---|---|
| Met | 1.47 |
| Glu | 1.44 |
| Leu | 1.30 |
| Ala | 1.29 |
| Gln | 1.27 |
| Lys | 1.23 |
| His | 1.22 |
| Cys | 1.11 |
| Phe | 1.07 |
| Asp | 1.04 |
| Trp | 0.99 |
| Ile | 0.97 |
| Arg | 0.96 |
| Val | 0.91 |
| Asn | 0.90 |
| Thr or Ser | 0.82 |
| Tyr | 0.72 |
| Gly | 0.56 |
| Pro | 0.52 |

The substitution of any amino acid for an amino acid with a higher frequency of occurrence in an alpha helix (as provided in the above table) would be a preferred substitution of the invention. More preferred are substitutions with an amino acid with a significantly higher frequency than the amino acid being substituted. The selection of any possible substitution, however, is preferably made in combination with other considerations known to, or desired by, the skilled person. As a non-limiting example, substitution with a leucine residue may not be as preferred as substitution with an alanine residue if the overall hydrophobicity of the peptide is preferably minimized.

Formulation and Use

The peptides of the invention may be used in a manner analogous to the use of any agent for the treatment of hair loss; for the treatment of psoriasis; and for the treatment of unwanted hair. Of course the peptides of the invention may be used singly or in combination with each other or other agents known in the art. Preferably, the compounds are administered in an effective amount such that an effect sufficient to stimulate hair growth, inhibit hair growth, or inhibit psoriasis as disclosed herein may occur. Repeated administration over time is within the scope of the present invention.

The peptides of the invention are preferably applied to skin topically. The peptides are preferably used to prepare a medicament, such as by formulation into pharmaceutical compositions for administration to a subject using techniques generally known in the art. A summary of such pharmaceutical compositions may be found, for example, in Remington's Pharmaceutical Sciences, Mack Publishing Co., Easton, Pa. The compounds of the invention can be used singly or as components of mixtures. A preferred form of the compounds is as a topical, or transdermal, formulation for application to human skin, although systemic administration may also be used. Formulations designed for timed release are also with the scope of the invention. Formulation in unit dosage form is also preferred for the practice of the invention.

The peptides of the invention may also be in the form of non-toxic salts, such as, but not limited to, salts resulting from addition of inorganic or organic acids. Non-limiting examples include hydrochloric acid, sulfuric acid, phosphoric acid, nitric acid, acetic acid, oxalic acid, tartaric acid, succinic acid, maleic acid, fumaric acid, gluconic acid, citric acid, malic acid, ascorbic acid, benzoic acid, polyglutamic acid, and the like. Salts of peptides resulting from the addition of base are also within the scope of the invention. Non-limiting examples include those formed with metal cations like zinc, calcium, magnesium, aluminum, cadmium, and the like.

The peptides of the invention may be labeled isotopically (e.g. with a radioisotope) or by another other means, including, but not limited to, the use of chromophores or fluorescent moieties, bioluminescent labels, or chemiluminescent labels. The compositions may be in conventional forms, either as liquid solutions or suspensions, solid forms suitable for solution or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. Of course, these compositions may also contain minor amounts of nontoxic, auxiliary substances, such as wetting or emulsifying agents, pH buffering agents, and so forth.

The peptides may be administered topically using standard topical compositions, such as lotions, suspensions, or pastes. Such compositions may comprise suitable carriers including thickeners, emollients, solvents, humectants, powders, and combinations thereof. Non-limiting examples of emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, mineral oil, butyl myristate, isostearic acid, palmitic acid, isopropyl linoleate, lauryl lactate, myristyl lactate, decyl oleate, and myristyl myristate. Non-limiting examples of solvents include ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, and tetrahydrofuran. Non-limiting examples of humectants include glycerin, sorbitol, sodium 2-pyrrolidone-5-carboxylate, soluble collagen, dibutyl phthalate, and gelatin. Non-limiting examples of powders include chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate.

The peptides of the invention may also be used with penetration enhancers that increase penetration of a peptide to the environment of the hair follicle. Non-limiting examples are D-limonene (1-20% v/v), Azone (0.0 to 10% v/v), N-methyl-2-pyrrylodione (1-20% v/v), sodium taurocholate (0.1 to 10% v/v), bile salt, ethanol, and propylene glycol. Other non-limiting examples of penetration enhancers include example, 2-methyl propan-2-ol, propan-2-ol, ethyl-2-hydroxypropanoate, hexan-2,5-diol, POE(2) ethyl ether, di(2-hydroxypropyl)ether, pentan-2,4-diol, acetone, POE(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, POE ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, iso-propyl palmitate, ethyl laurate, 2-ethyl-hexyl pelargonate, iso-propyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, and 1-dodecylazacyloheptan-2-one.

The appropriate peptides of the invention may also be used in combination with a hair growth activity enhancer, such as, but not limited to, benzalkonium chloride, benzethonium chloride, phenol, estradiol, diphenylhydramine hydrochloride, chlorpheniramine maleate, chlorophyllin derivatives, cholesterol, salicylic acid, cysteine, methionine, red pepper tincture, benzyl nicotinate, D,L-menthol, peppermint oil, calcium pantothenate, panthenol, castor oil, hinokitiol, prednisolone, resorcinol, monosaccharides and esterified monosaccharides, chemical activators of protein kinase C enzymes, glycosaminoglycan chain cellular uptake inhibitors, inhibitors of glycosidase activity, glycosaminoglycanase inhibitors, esters of pyroglutamic acid, hexosaccharic acids or acylated hexosaccharic acids, aryl-substituted ethylenes, N-acylated amino acids, Propecia™ (finasteride), and minoxidil.

The peptides of the invention may also be administered by iontophoresis. See, e.g., Banga et al., “Hydrogel-based Iontotherapeutic Delivery Devices for Transdermal Delivery of Peptide/Protein Drugs”, Pharm. Res., Vol. 10 (5), pp. 697-702 (1993); Ferry, “Theoretical Model of Iontophoresis Utilized in Transdermal Drug Delivery”, Pharmaceutical Acta Helvetiae, Vol 70, pp. 279-287 (1995); Gangarosa et al., “Modern Iontophoresis for Local Drug Delivery”, Int. J. Pharm, Vol. 123, pp. 159-171 (1995); Green et al., “Iontophoretic Delivery of a Series of Tripeptides Across the Skin in vitro”, Pharm. Res., Vol 8, pp. 1121-1127 (1991); Jadoul et al., “Quantification and Localization of Fentanyl and TRH Delivered by Iontophoresis in the Skin”, Int. J. Pharm., Vol. 120, pp. 221-8 (1995); Parry et al., “Acyclovir Biovailability in Human Skin”, J. Invest. Dermatol., Vol. 98 (6), pp. 856-63 (1992); Santi et al., “Drug Reservoir Composition and Transport of Salmon Calcitonin in Transdermal Iontophoresis”, Pharm. Res., Vol 14 (1), pp. 63-66 (1997); Santi et al., “Reverse Iontophoresis—Parameters Determining Electroosmotic Flow: I. pH and Ionic Strength”, J. Control. Release, Vol. 38, pp. 159-165 (1996); Santi et al., “Reverse Iontophoresis—Parameters Determining Electroosmotic Flow: II. Electrode Chamber Formulation”, J. Control. Release, Vol. 42, pp. 29-36 (1996); Rao et al., “Reverse Iontophoresis: Noninvasive Glucose Monitoring in vivo in Humans”, Pharm. Res., Vol. 12 (12), pp. 1869-1873 (1995); Thysman et al., “Human Calcitonin Delivery in Rats by Iontophoresis”, J. Pharm. Pharmacol., Vol. 46, pp. 725-730 (1994); and Volpato et al., “Iontophoresis Enhances the Transport of Acyclovir through Nude Mouse Skin by Electrorepulsion and Electroosmosis”, Pharm. Res., Vol. 12 (11), pp. 1623-1627 (1995).

Topical compositions of the present invention may be in any form including, for example, solutions, oils, creams, ointments, gels, lotions, shampoos, leave-on and rinse-out hair conditioners, milks, cleansers, moisturizers, sprays, skin patches, and the like.

The quantity of the peptide to be administered depends upon the active thereof the condition to be treated, the mode of administration, the individual subject, and the judgment of the practitioner. Depending on the specificity of the preparation, smaller or larger doses may be needed. For compositions that are highly active, dosages in the range of about 0.01 μg/kg to about 1 mg/kg body weight are suggested, although dosages from about 0.05 to about 0.2 μg/kg, about 0.2 to about 1 μg/kg, about 1 to about 10 μg/kg, about 10 to about 100 μg/kg, or about 100 to about 500 μg/kg body weight may also be used. Particularly preferred for the practice of the invention are solutions of about 1 μg to about 100 mg per milliliter, more preferably about 10 μg to about 75 mg, about 100 μg to about 50 mg, about 1 mg to about 25 mg, or about 10 mg per milliliter of solution. Particularly preferred is the use of about a 50 mg/ml solution, which is approximately a 5% solution, although solutions of about 1 to about 5% or about 5 to about 10% may also be formulated and used. The inclusion of penetration or activity enhancers will of course permit the use of lower concentrations of peptide. For compositions that are less active, larger doses, up to 1-10 mg/kg may be needed.

The foregoing ranges are merely suggestive, as the number of variables in regard to an individual treatment regime is large, and considerable excursions from these recommended values are not uncommon.

While the invention has been detailed mainly with respect to the treatment of human beings, preferred animals for the application of the present invention are mammals, particularly those important to agricultural applications (such as, but not limited to, cattle, sheep, horses, and other “farm animals”) and zoological conservation efforts as well as for human companionship (such as, but not limited to, dogs and cats).

Methods of Preparation

The peptides of the invention may be produced by solid or liquid phase synthesis as described herein. Methods for the production by such methods are known in the art, as well as methods for the purification of peptides produced by such methods.

For the preparation of a peptide by recombinant expression, the present invention provides for a polynucleotide encoding a peptide of the invention as well as vectors containing said polypeptide. Such vectors are capable of expressing said peptide, and may be used to do so via in vitro (cell free) transcription and/or translation methods or by introduction into suitable host cells. Introduction of vectors of the invention into host cells can be conducted by a variety of methods known to the skilled person. Non-limiting examples include calcium phosphate transfection, DEAE-dextran mediated transfection, microinjection, cationic lipid-mediated transfection, electroporation, transduction, or infection with a viral vector.

Suitable host cells for the expression of peptides include both prokaryotic and eukaryotic cells. Non-limiting examples of prokaryotic cells are E. coli, Streptomyces and Bacillus subtilis cells while such examples of eukaryotic cells are fungal cells, insect cells, and animal cells such as CHO, COS, HeLa, C127, 3T3, BHK, and 293 cells. In other preferred embodiments of the invention, plant cells or whole plants may be used to recombinantly produce peptides of the invention.

The selection of a suitable and compatible vector/host cell system is known in to the skilled person. The vector may be viewed as an expression system and may be of a variety of types, including, but not limited to, chromosomal, episomal and viral based systems. Plasmids which are maintained episomally or in an integrated form are one preferred means of practicing the invention. An expression system comprises control region(s) that regulate the expression of a coding sequence operably linked thereto while also providing a ready means to propagate said coding sequence. Routine methods for the introduction of a coding sequence into a vector are known and may be used in the practice of the invention to produce a vector that maintains, propagates or expresses a coding sequence to produce a peptide of the invention in a host cell.

The term “operably linked” refers to a linkage in which the control or regulatory DNA sequences (usually a promoter and sequences that, upon expression, direct the initiation of translation) and the coding sequence to be expressed are connected in such a way as to permit expression of the intended peptide. The control or regulatory region for use in various cells varies with the choice of said cell as known to the skilled person. For eukaryotic cells, sequences that regulate mRNA processing, transport, stability and use may also used to in an operable linkage in effecting the expression of a peptide of the invention.

In an alternative embodiment, RNA vectors may also be utilized to express a peptide of the invention. Such vectors are based on positive or negative strand RNA viruses as known to the skilled person.

To have a peptide of the invention secreted into the lumen of the endoplasmic reticulum, into the periplasmic space or into the extracellular environment after (or during) translation, sequences encoding an appropriate signal may be fused to the coding sequence of the peptide to result in the expression of a fusion polypeptide comprising the signal and said peptide. Such a signal may be endogenous to the peptide or may be a heterologous signal.

Activities and Assays

As disclosed herein, the peptides of the invention are useful for the treatment of conditions associated with hair growth/loss and psoriasis of the skin. The activities of the peptides are based upon their abilities to inverse agonize or agonize the PTH1R receptor. An “agonist” peptide is capable of enhancing, increasing, or potentiating a PTH1R receptor mediated response while an “antagonist” peptide is capable of decreasing or inhibiting a PTH1R receptor mediated response. As noted above, an “inverse agonist” peptide reverses the activity of the receptor. Whether, and to what extent, a peptide of the invention acts as an “agonist” or “antagonist” or “inverse agonist” can be determined using art-known protein ligand/receptor cellular response or binding assays.

Such assays may be based upon a variety of activities, including, but not limited to, binding to the PTH1R receptor (to determine whether any PTH1R mediated activity is possible); a cAMP accumulation assay (where native PTH activates cAMP accumulation in a cell such that peptides that decrease such accumulation are antagonists and peptides that decrease cAMP levels are inverse agonists); an intracellular calcium level increase assay (where an agonist increases intracellular calcium, an antagonists blocks such increases, and an inverse agonist decreases intracellular calcium); stimulation of hair growth or inhibition of hair loss; inhibition of hair growth or stimulation of hair loss; and inhibition of psoriasis.

The following examples are put forth so as to provide those of ordinary skill in the art with a complete disclosure and description of how to make and use the present invention, and are not intended to limit the scope of what is regarded as the invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLE 1

### General Procedures

The peptides of the invention were synthesized via liquid phase peptide chemistry and mass spectroscopy (MS) was used to determine purity (>=90%). The peptides were dissolved in 10 mM acetic acid, with the concentration calculated using the peptide content and weight provided by in lab analysis. Aliquots were stored at −80° C. and used once. Lactose peroxidase was obtained from Sigma. Cell culture supplies were obtained from Life Technologies, Inc. except for Dulbecco's modified Eagle's medium, which was from Mediatech (Herndon, Va.).

### EXAMPLE 2

### Preparation of Radioligands

Radioligand forms of the peptides of the invention were prepared using chloramine T as catalyst and the di-iodinated peptide (6000 Ci/mmol) purified by HPLC. The peptides were processed using the lactose-peroxidase method. 10 mg in 10 ml of reaction buffer (0.1 M sodium acetate buffer, pH 6.5) was dispensed into a siliconized microcentrifuge tube, followed by the sequential addition of 0.5 mCi of Na¹²⁵I, 10 ml of 40 mg/ml lactose peroxidase in reaction buffer, and 90 ml of reaction buffer. After mixing, 10 ml of 0.001% H₂O₂ was added. After 20 min at room temperature, the reaction was terminated by the addition of 0.5 ml of reaction buffer supplemented with 0.1% sodium azide. After an additional 5 min, 0.5 ml of reaction buffer supplemented with 1 M NaCl, 0.1% bovine serum albumin, and 1% potassium iodide was added. The radioligand was then desalted using a C18 cartridge and purified by high pressure liquid chromatography. The radioactive peak fractions corresponded with a single peak of UV absorbance.

### EXAMPLE 3

### Radioligand Binding Assay

In these assays, the binding of a range of concentrations of an unlabeled ligand was measured by displacement of radioligand binding. Three methods were employed. An assay employing centrifugation to separate bound and free radioligand was used to accurately measure ligand binding parameters. A higher through-put method employing rapid filtration was used to generate comparative ligand binding data. Whole-cell binding assays were used to measure radioligand binding to chimeric PTH2/glucagons receptors, since this assay provides the highest total binding/nonspecific binding ratio, important for detecting lower affinity binding of radioligands. In all these assays, a very low concentration of radioligand was used so that the IC₅₀ closely approximates the ligand affinity.

In the centrifugation assay, cell membranes (45-50 mg), radioligand (100,000-300,000 cpm), and unlabeled ligand were incubated in a final volume of 1 ml of assay buffer (20 mM HEPES, 100 mM NaCl, 1 mM EDTA, 3 mM MgSO₄, pH 7.5, supplemented with 0.3% nonfat dried milk powder, 100 mM (4-(2-aminoethyl))-benzenesulfonylfluoride, and 1 mg/ml bacitracin) for 2 hours at 21° C. Membranes were collected at 18,000×g, the surface of the pellet was gently washed, and the radioactivity was counted. For the PTH1 receptor, ¹²⁵I labeled peptides (sequences) were used as radioligand at a final concentration of approximately 20-32 pM.

Membranes were harvested via a standardized protocol. Total binding was less than 15% of the total amount of radioactivity added. Whole-cell binding assay studies were also performed.

### EXAMPLE 4

### Methods for Cell Culture and Expression in COS-7 Cells

The assay is an already accepted model using COS-7 cells which were grown as previously described by Clark J A, Bonner T I, Kim A S, Usdin T B (Multiple regions of ligand discrimination revealed by analysis of chimeric parathyroid hormone 2 (PTH2) and PTH/PTH-related peptide (PTHrP) receptors. Mol Endocrinol 1998;12:193-206). The COS-7 cells were also transfected as previously described therein, using 10 cm tissue culture dishes and 10 micrograms of plasmid DNA.

At 24 hours the cells were transferred following trypsinization (edeate) to 96-well plates at a density of approx. 50,000 cells per well.

ROS 17/2.8 cells were grown in tissue-culture flasks in Ham's F-12 medium, 5% fetal bovine serum, 100 U/ml penicillin and 100 μg/ml streptomycin. For cAMP accumulation assays ROS 17/2.8 cells were transferred 2 days prior to assay into 96-well plates at 20,000 cells per well.

### EXAMPLE 5

### Data Analysis

Concentration dependence data for ligand-stimulated cAMP accumulation and displacement of radioligand binding were analyzed using the following four-parameter logistic equation using:

Prism 2.01 (GraphPad Software Inc., San Diego, Calif.), y 5 min 1˜max 2 min!/˜11 10˜logK2X!nH! where X represents the logarithm of the ligand concentration and nH represents the pseudo-Hill slope.

For cAMP accumulation, y represents the amount of cAMP produced at a given peptide concentration, min is the cAMP level in the absence of ligand, and max is the maximum level produced. For inhibition of radioligand binding, y is the cpm bound at a given unlabeled ligand concentration, min is nonspecific binding (measured in the presence of a high concentration of the unlabeled version of the radiolabeled ligand), and max is total binding (measured in the absence of unlabeled ligand). Comparison of multiple means was performed initially by single factor analysis of variance followed by post hoc analysis with the Newman test. Statistical comparison of two means was performed using a two-tailed Student's test.

### EXAMPLE 6

### Other Assays

The above binding and cAMP accumulation assays, as well as assays for increases in intracellular calcium levels; stimulation of hair growth or inhibition of hair loss; inhibition of hair growth or stimulation of hair loss; and inhibition of psoriasis may be used to determine the level of inverse agonist, antagonist, or agonist activity in a peptide of the invention. The assay may be cell free, as in the case of binding assays, or utilize living cells or animals (expressing a PTH1R receptor or the counterpart thereof in another cell type or animal species), as in the case of assays related to cAMP, calcium levels, hair growth, or psoriasis. In all assays, the use of a first amount or concentration of a peptide of the invention, in comparison to the absence of said peptide or the use of a second (different) amount or concentration provides a means to determine the level of activity by said first amount or concentration.

As a non-limiting example of a assay based on increases in intracellular calcium, the invention provides for a method comprising

a) providing a population of cells;

b) contacting one or more cells of said population with a first amount or concentration of a peptide of the invention to produce a first contacted cell or cells and contacting a second cell or cells of said population with a second amount or concentration of said peptide to produce a second contacted cell or cells; and

c) determining the level of intracellular calcium in said first contacted cell or cells and in said second contacted cell or cells,

wherein the difference in intracellular calcium levels between said first and second contacted cell or cells indicates the level of activity by said peptide on cellular calcium levels. The first or second amount or concentration of a peptide may of course be zero such that the peptide is absent. The population of cells are those known or used for assays for intracellular calcium based on the PTH1R receptor.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A peptide comprising the amino acid sequence represented by Formula 1:

| | |
|---|---|
| X1-X2-His-Asn-X5-X6-X7-X8-X9-X10- | (SEQ ID NO: 4) |
| X11-X12-X13-Arg-X15-X16-X17-Leu-X19- | |
| X20-X21-X22-X23-X24-X25-His-X27-X28 | |

wherein X1 is independently leucine or phenylalanine; X2 is independently methionine, leucine, or norleucine; X5 is independently leucine or lysine; X6 is independently glycine, D-tryptophan, D-α-Nal, or D-β-Nal; X7 is lysine, optionally substituted at its epsilon amino group; X8 is independently serine or histidine; X9 is independently leucine or isoleucine; X10 is independently asparagine, glutamine, serine or alanine; X11 is independently serine or aspartic acid; X12 is independently methionine or leucine, norleucine or valine; X13 is independently glutamic acid or arginine; X15 is independently valine, arginine or methionine; X16 is independently glutamic acid, phenylalanine or glutamine; X17 is independently tryptophan or phenylalanine; X19 is independently arginine or histidine; X20 is independently lysine or histidine; X21 is independently lysine or leucine; X22 is independently leucine or isoleucine; X23 is independently glutamine or alanine; X24 is independently aspartic acid or glutamic acid; X25 is independently valine or isoleucine; X27 is independently asparagine or threonine; and X28 is independently phenylalanine, tyrosine, or alanine; and
wherein said peptide is not human or bovine PTHrP 7-34.

2. The peptide of claim 1 wherein X11 is an aspartic residue that is cyclized with the lysine residue at X7.

3. The peptide of claim 1 wherein X16 is a glutamic acid residue that is cyclized with a lysine residue at position X20.

4. The peptide of claim 1 wherein X20 is a histidine residue that is cyclized with a glutamic acid residue at position X24.

5. The peptide of claim 1 wherein X11 is an aspartic residue that is cyclized with the lysine residue at X7 and either
a) X16 is a glutamic acid residue that is cyclized with a lysine residue at position X20; or
b) X20 is a histidine residue that is cyclized with a glutamic acid residue at position X24.

6. The peptide of claim 1 wherein X2 and X12 are independently leucine or norleucine.

7. The peptide of claim 1 wherein the sequence represented by Formula 1 is selected from

| | | |
|---|---|---|
| a) | Leu-Leu-His-Asn-Leu-Gly-Lys- | (SEQ ID NO: 2) |
| | Ser-Ile-Gln-Asp-Leu-Arg-Arg- | |
| | Arg-Phe-Phe-Leu-His-His-Leu- | |
| | Ile-Ala-Glu-Ile-His-Thr-Ala; | |
| b) | Leu-Leu-His-Asn-Leu-D-Trp- | (SEQ ID NO: 5) |
| | Lys-Ser-Ile-Gln-Asp-Leu-Arg- | |
| | Arg-Arg-Phe-Phe-Leu-His-His- | |
| | Leu-Ile-Ala-Glu-Ile-His-Thr- | |
| | Ala; | |
| c) | Leu-Leu-His-Asp-Leu-D-Trp- | (SEQ ID NO: 5) |
| | Lys-Ser-Ile-Gln-Asp-Leu-Arg- | |
| | Arg-Arg-Phe-Phe-Leu-His-His- | |
| | Leu-Ile-Ala-Glu-Ile-His-Thr- | |
| | Ala; | |
| d) | Phe-Met-His-Asn-Leu-Gly-Lys- | (SEQ ID NO: 6) |
| | His-Leu-Ser-Ser-Met-Glu-Arg- | |
| | Val-Glu-Trp-Leu-Arg-Lys-Lys- | |
| | Leu-Gln-Asp-Val-His-Asn-Tyr; | |
| e) | Phe-Nle-His-Asn-Leu-Gly- | (SEQ ID NOS: 7-8) |
| | Lys_{(epsilon-3-phenylpro-} | |
| | _{panoyl)}-His-Leu-Ser-Ser-Nle- | |
| | Glu-Arg-Val-Glu-Trp-Leu-Arg- | |
| | Lys-Lys-Leu-Gln-Asp-Val-His- | |
| | Asn-Tyr; and | |
| f) | Leu-Leu-His-Asp-Leu-D-Trp- | (SEQ ID NO: 10) |
| | Lys(N epsilon-_{(biotinyl-beta-} | |
| | _{Ala))}-Ser-Ile-Gln-Asp-Leu- | |
| | Arg-Arg-Arg-Phe-Phe-Leu-His- | |
| | His-Leu-Ile-Ala-Glu-Ile-His- | |
| | Thr-Ala. | |

8. A peptide comprising the amino acid sequence represented by Formula 5:

| | |
|---|---|
| Asp-Ala-Ala-Phe-Arg-Glu-Arg- | (SEQ ID NO: 25) |
| Ala-Arg-Leu-Leu-Ala-Ala-Leu- | |
| Glu-Arg-Arg-X18-Trp-Leu-X21- | |
| Ser-Tyr-X24-X25-Lys-Leu-Leu- | |
| X29-Leu-Asp-Ala-Pro | |

wherein X18 is independently histidine or arginine; X21 is independently aspartic acid or asparagine; X24 is independently methionine or norleucine; X25 is independently glutamine or histidine; and X29 is independently leucine or valine; and
wherein said sequence is not that of human/bovine TIP 7-39.

9. The peptide of claim 8 wherein the sequence represented by Formula 5 is selected from

| | |
|---|---|
| Asp-Ala-Ala-Phe-Arg-Glu-Arg- | (SEQ ID NO: 24) |
| Ala-Arg-Leu-Leu-Ala-Ala-Leu- | |
| Glu-Arg-Arg-Arg-Trp-Leu-Asp- | |
| Ser-Tyr-Met-Gln-Lys-Leu-Leu- | |
| Leu-Leu-Asp-Ala-Pro; | |
| Asp-Ala-Ala-Phe-Arg-Glu-Arg- | (SEQ ID NO: 26) |
| Ala-Arg-Leu-Leu-Ala-Ala-Leu- | |
| Glu-Arg-Arg-His-Trp-Leu-Asn- | |
| Ser-Tyr-Nle-His-Lys-Leu-Leu- | |
| Val-Leu-Asp-Ala-Pro; | |
| Asp-Ala-Ala-Phe-Arg-Glu-Arg- | (SEQ ID NO: 27) |
| Ala-Arg-Leu-Leu-Ala-Ala-Leu- | |
| Glu-Arg-Arg-His-Trp-Leu-Asn- | |
| Ser-Tyr-Met-His-Lys-Leu-Leu- | |
| Val-Leu-Asp-Ala-Pro; | |
| Asp-Ala-Ala-Phe-Arg-Glu-Arg- | (SEQ ID NO: 24) |
| Ala-Arg-Leu-Leu-Ala-Ala-Leu- | |
| Glu-Arg-Arg-Arg-Trp-Leu-Asp- | |
| Ser-Tyr-Met-Gln-Lys-Leu-Leu- | |
| Leu-Leu-Asp-Ala-Pro; and | |
| Asp-Ala-Ala-Phe-Arg-Glu-Arg- | (SEQ ID NO: 28) |
| Ala-Arg-Leu-Leu-Ala-Ala-Leu- | |
| Glu-Arg-Arg-Arg-Trp-Leu-Asp- | |
| Ser-Tyr-Nle-Gln-Lys-Leu-Leu- | |
| Leu-Leu-Asp-Ala-Pro. | |

10. A pharmaceutical composition comprising the peptide of claim 1 and a pharmaceutically acceptable excipient.

11. The composition of claim 10 formulated for topical application.

12. A method of stimulating hair growth comprising administration of the peptide of claim 1.

13. A peptide comprising the amino acid sequence represented by Formula 6:

| | |
|---|---|
| X1-Val-X3-Glu-Ile-Gln- | (SEQ ID NO: 30) |
| Leu-X8-His-X10-X11 | |

wherein X1 and X3 are independently serine, alanine, or a-aminoisobutyric acid (Aib); X8 is independently methionine or norleucine; X10 is independently asparagine, alanine, glutamine, or histidine; and X11 is independently leucine, arginine, or homoarginine.

14. The peptide of claim 13 comprising the amino acid sequence represented by the formula

| | |
|---|---|
| X1-Val-X3-Glu-Ile-Gln-Leu- | (SEQ ID NO: 31) |
| X8-His-X10-X11-X12-Lys-X14 | |

wherein X12 is independently glycine or alanine; and X14 is independently histidine or tryptophan; and wherein said peptide is not amino acids 1-14 of human PTH.

15. The peptide of claim 13 wherein the sequence represented by Formula 6 is selected from

| | |
|---|---|
| Ser-Val-Ser-Glu-Ile-Gln-Leu-Met- | (SEQ ID NO: 32) |
| His-Asn-Leu; | |
| Ser-Val-Ser-Glu-Ile-Gln-Leu-Met- | (SEQ ID NO: 33) |
| His-His-Leu; | |
| Ser-Val-Ala-Glu-Ile-Gln-Leu-Met- | (SEQ ID NO: 34) |
| His-Gln-Har; | |
| Aib-Val-Aib-Glu-Ile-Gln-Leu-Met- | (SEQ ID NO: 35) |
| His-Gln-Har-Ala-Lys-Trp; | |
| Ser-Val-Ser-Glu-Ile-Gln-Leu-Met- | (SEQ ID NO: 36) |
| His-His-Leu-Gly-Lys-His; | |
| Ser-Val-Ala-Glu-Ile-Gln-Leu-Met- | (SEQ ID NO: 37) |
| His-Gln-Har-Ala-Lys-Trp; | |
| Ser-Val-Ala-Glu-Ile-Gln-Leu-Met- | (SEQ ID NO: 38) |
| His-Ala-Arg-Ala-Lys-Trp; and | |
| Ser-Val-Ala-Glu-IIe-Gln-Leu-Met- | (SEQ ID NO: 39) |
| His-His-Arg-Ala-Lys-Trp. | |

16. A peptide comprising the amino acid sequence represented by Formula 7:

| | |
|---|---|
| X1-Val-X3-Glu-Ile-Gln-X7-X8- | (SEQ ID NO: 40) |
| His-X10-X11-X12-Lys-X14-X15- | |
| X16-X17-X18-X19-Arg-X21-X22- | |
| X23-Leu-X25-X26-X27-X28-X29- | |
| X30-X31-His-X33-X34 | |

wherein X1 and X3 are independently serine or alanine; X7 is independently leucine or phenylalanine; X8 is independently methionine, leucine, or norleucine; X10 is independently asparagine, glutamine, aspartic acid, or histidine; X11 is independently leucine, lysine, or homoarginine; X12 is independently glycine or alanine; X14 is independently histidine, serine, or tryptophan; X15 is independently leucine or isoleucine; X16 is independently asparagine, alanine, serine or glutamine; X17 is independently serine or aspartic acid; X18 is methionine, norleucine, valine, or leucine; X19 is glutamic acid or arginine; X21 is independently valine, methionine, norleucine, or arginine; X22 is independently glutamic acid, glutamine, isoleucine, or phenylalanine; X23 is independently tryptophan or phenylalanine; X25 is independently arginine, glutamine, or histidine; X26 is independently lysine, asparagine, or histidine; X27 is independently lysine or leucine; X28 is independently leucine or isoleucine; X29 is independently glutamine, glutamic acid, or alanine; X30 is independently aspartic acid, glycine, or glutamic acid; X31 is independently valine or isoleucine; X33 is independently asparagine or threonine; and X34 is independently phenylalanine, tyrosine, or alanine;
and wherein said peptide is not human PTH.

17. The peptide of claim 16 wherein the sequence represented by Formula 7 is selected from

| | |
|---|---|
| Ala-Val-Ser-Glu-Ile-Gln-Phe- | (SEQ ID NO: 41) |
| Nle-His-Asn-Leu-Gly-Lys-His- | |
| Leu-Ser-Ser-Nle-Glu-Arg-Val- | |
| Glu-Trp-Leu-Arg-Lys-Lys-Leu- | |
| Gln-Asp-Val-His-Asn-Tyr; | |
| Ala-Val-Ser-Glu-Ile-Gln-Leu- | (SEQ ID NO: 42) |
| Nle-His-Asn-Leu-Gly-Lys-His- | |
| Leu-Ala-Ser-Val-Glu-Arg-Nle- | |
| Gln-Trp-Leu-Arg-Lys-Lys-Leu- | |
| Gln-Asp-Val-His-Asn-Tyr; | |
| Ala-Val-Ser-Glu-Ile-Gln-Phe- | (SEQ ID NO: 43) |
| Met-His-Asn-Leu-Gly-Lys-His- | |
| Leu-Ser-Ser-Met-Glu-Arg-Val- | |
| Glu-Trp-Leu-Arg-Lys-Lys-Leu- | |
| Gln-Asp-Val-His-Asn-Tyr; | |
| Ala-Val-Ser-Glu-Ile-Gln-Phe- | (SEQ ID NO: 44) |
| Nle-His-Asn-Leu-Gly-Lys-His- | |
| Leu-Ser-Ser-Nle-Glu-Arg-Val- | |
| Glu-Trp-Leu-Arg-Lys-Lys-Leu- | |
| Gln-Asp-Val-His-Asn-Tyr; | |
| Ser-Val-Ser-Glu-Ile-Gln-Leu- | (SEQ ID NO: 45) |
| Nle-His-Asn-Leu-Gly-Lys-His- | |
| Leu-Asn-Ser-Nle-Glu-Arg-Val- | |
| Glu-Trp-Leu-Arg-Lys-Lys-Leu- | |
| Gln-Asp-Val-His-Asn-Tyr; | |
| Ala-Val-Ser-Glu-Ile-Gln-Leu- | (SEQ ID NO: 46) |
| Met-His-Asn-Leu-Gly-Lys-His- | |
| Leu-Ala-Ser-Val-Glu-Arg-Nle- | |
| Gln-Trp-Leu-Arg-Lys-Lys-Leu- | |
| Gln-Asp-Val-His-Asn-Phe; | |
| Ala-Val-Ser-Glu-His-Gln-Leu- | (SEQ ID NO: 47) |
| Leu-His-Asp-Lys-Gly-Lys-Ser- | |
| Ile-Gln-Asp-Leu-Arg-Arg-Arg- | |
| Phe-Phe-Leu-His-His-Leu-Ile- | |
| Ala-Glu-Ile-His-Thr-Tyr; and | |
| Ala-Val-Ser-Glu-His-Gln-Leu- | (SEQ ID NO: 48) |
| Leu-His-Asp-Lys-Gly-Lys-Ser- | |
| Ile-Gln-Asp-Leu-Arg-Arg-Arg- | |
| Ile-Phe-Leu-Gln-Asn-Leu-Ile- | |
| Glu-Gly-Val-Asn-Thr-Ala-Glu- | |
| Tyr. | |

18. A non-naturally occurring peptide comprising the amino acid sequence represented by Formula 4:

| | |
|---|---|
| X1-X2-X3-X4-X5-X6-Lys-His/Ser-Leu/Ile-Ser/Asn/Gln/Ala-Ser/Asp-Met/Leu/Nle/Val- | (SEQ ID NO:22) |
| Arg-Arg-X15-Glu-X17-Leu-X19-Lys-X21-X22-X23-X24-X25-His-X27-X28 | |

wherein the X5-X6-Lys residues form a turn of about 15 degrees and positions X1 to X4 and Arg-X15-Glu-X17-Leu-X19-Lys-X21-X22-X23-X24-X25-His-X27-X28 (portion of SEQ ID NO:22)form alpha helical structures composed of naturally occurring amino acids; and
wherein the Lys residue in X5-X6-Lys is optionally substituted.

19. The peptide of claim 18 wherein X5-X6 is Lys-Gly, Leu-Gly, Lys-D-Trp, Leu-D-Trp, Lys-D-α-Nal, Leu-D-α-Nal, Lys-D-β-Nal, or Leu-D-β-Nal.

20. A pharmaceutical composition comprising the peptide of claim 13 and a pharmaceutically acceptable excipient.

21. The composition of claim 20 formulated for topical application.

22. A method of inhibiting hair growth comprising administration of the peptide of claim 13.

23. A method of inhibiting psoriasis comprising administration of the peptide of claim 13.

24. A method of assaying a peptide of claim 1 to determine the level of inverse agonist activity against the PTH1R receptor comprising
a) detection of binding to PTH1R;
b) detection of cAMP accumulation or intracellular calcium increases;
c) stimulation of hair growth or inhibition of hair loss;
d) inhibition of hair growth or stimulation of hair loss; and
inhibition of psoriasis.


SEQUENCE LISTING (48 sequences)

SEQ ID NO: 1 - protein, 28 aa, Homo sapiens
LMHNLGKHLNSMERVEWLRKKLQDVHNF

SEQ ID NO: 2 - protein, 28 aa, Homo sapiens
LLHDKGKSIQDLRRRFFLHHLIAEIHTA

SEQ ID NO: 3 - protein, 28 aa, Homo sapiens
FMHNLGKHLSSMERVEWLRKKLQDVHNF

SEQ ID NO: 4 - protein, 28 aa, Homo sapiens
  VARIANT 1, 2, 5, 6, 7, 8, 9, 10, 11, 12, 13, 15, 16, 17, 19, 20,21, 22, 23, 24, 25, 27, 28: Xaa = Any Amino Acid
XXHNXXXXXXXXXRXXXLXXXXXXXHXX

SEQ ID NO: 5 - protein, 28 aa, Homo sapiens
LLHNLWKSIQDLRRRFFLHHLIAEIHTA

SEQ ID NO: 6 - protein, 28 aa, Homo sapiens
FMHNLGKHLSSMERVEWLRKKLQDVHNY

SEQ ID NO: 7 - protein, 7 aa, Homo sapiens
  MOD_RES 2: Nle
FXHNLGK

SEQ ID NO: 8 - protein, 21 aa, Homo sapiens
  MOD_RES 5: Nle
HLSSXERVEWLRKKLQDVHNY

SEQ ID NO: 9 - protein, 7 aa, Homo sapiens
LLHDLWK

SEQ ID NO: 10 - protein, 21 aa, Homo sapiens
SIQDLRRRFFLHHLIAEIHTA

SEQ ID NO: 11 - protein, 28 aa, Homo sapiens
  VARIANT 1, 2, 5, 6, 8, 9, 10, 12, 13, 15, 16, 17, 19, 20, 21,22, 23, 24, 25, 27, 28: Xaa = Any Amino Acid
XXHNXXKXXXDXXRXXXLXXXXXXXHXX

SEQ ID NO: 12 - protein, 7 aa, Homo sapiens
LLHDLWK

SEQ ID NO: 13 - protein, 4 aa, Homo sapiens
SIQD

SEQ ID NO: 14 - protein, 17 aa, Homo sapiens
LRRRFFLHHLIAEIHTA

SEQ ID NO: 15 - protein, 28 aa, Homo sapiens
  VARIANT 1, 2, 5, 6, 7, 8, 9, 10, 11, 12, 13, 15, 17, 19, 21, 22,23, 24, 25, 27, 28: Xaa = Any Amino Acid
XXHNXXXXXXXXXRXEXLXKXXXXXHXX

SEQ ID NO: 16 - protein, 16 aa, Homo sapiens
LMHNLGKHLNSMERVE

SEQ ID NO: 17 - protein, 4 aa, Homo sapiens
WLRK

SEQ ID NO: 18 - protein, 8 aa, Homo sapiens
KLQDVHNF

SEQ ID NO: 19 - protein, 20 aa, Homo sapiens
LLHDKGKSIQDLRRRFFLHH

SEQ ID NO: 20 - protein, 4 aa, Homo sapiens
LIAE

SEQ ID NO: 21 - protein, 4 aa, Homo sapiens
IHTA

SEQ ID NO: 22 - protein, 28 aa, Homo sapiens
  VARIANT 1, 2, 3, 4, 5, 6, 8, 9, 10, 11, 12, 14, 15, 16, 17, 18,19, 20, 21, 22, 23, 24, 25, 26, 27, 28: Xaa = Any Amino Acid
XXXXXXKXXXXXRXXXXXXXXXXXXXXX

SEQ ID NO: 23 - protein, 33 aa, Homo sapiens
DAAFRERARLLAALERRHWLNSYMHKLLVLDAP

SEQ ID NO: 24 - protein, 33 aa, Homo sapiens
DAAFRERARLLAALERRRWLDSYMQKLLLLDAP

SEQ ID NO: 25 - protein, 33 aa, Homo sapiens
  VARIANT 18, 21, 24, 25, 29: Xaa = Any Amino Acid
DAAFRERARLLAALERRXWLXSYXXKLLXLDAP

SEQ ID NO: 26 - protein, 33 aa, Homo sapiens
  MOD_RES 24: Nle
DAAFRERARLLAALERRHWLNSYXHKLLVLDAP

SEQ ID NO: 27 - protein, 33 aa, Homo sapiens; identical to SEQ ID NO: 23

SEQ ID NO: 28 - protein, 33 aa, Homo sapiens
  MOD_RES 24: Nle
DAAFRERARLLAALERRRWLDSYXQKLLLLDAP

SEQ ID NO: 29 - protein, 34 aa, Homo sapiens
SVSEIQLMHNLGKHLNSMERVEWLRKKLQDVHNF

SEQ ID NO: 30 - protein, 11 aa, Homo sapiens
  VARIANT 1, 3, 8, 10, 11: Xaa = Any Amino Acid
XVXEIQLXHXX

SEQ ID NO: 31 - protein, 14 aa, Homo sapiens
  VARIANT 1, 3, 8, 10, 11, 12, 14: Xaa = Any Amino Acid
XVXEIQLXHXXXKX

SEQ ID NO: 32 - protein, 11 aa, Homo sapiens
SVSEIQLMHNL

SEQ ID NO: 33 - protein, 11 aa, Homo sapiens
SVSEIQLMHHL

SEQ ID NO: 34 - protein, 11 aa, Homo sapiens
SVAEIQLMHQR

SEQ ID NO: 35 - protein, 14 aa, Homo sapiens
  MOD_RES 1: Aib
  MOD_RES 3: Aib
XVXEIQLMHQRAKW

SEQ ID NO: 36 - protein, 14 aa, Homo sapiens
SVSEIQLMHHLGKH

SEQ ID NO: 37 - protein, 14 aa, Homo sapiens
SVAEIQLMHQRAKW

SEQ ID NO: 38 - protein, 14 aa, Homo sapiens
SVAEIQLMHARAKW

SEQ ID NO: 39 - protein, 14 aa, Homo sapiens
SVAEIQLMHHRAKW

SEQ ID NO: 40 - protein, 34 aa, Homo sapiens
  VARIANT 1, 3, 7, 8, 10, 11, 12, 14, 15, 16, 17, 18, 19, 21, 22,23, 25, 26, 27, 28, 29, 30, 31, 33, 34: Xaa = Any Amino Acid
XVXEIQXXHXXXKXXXXXXRXXXLXXXXXXXHXX

SEQ ID NO: 41 - protein, 34 aa, Homo sapiens
  MOD_RES 8: Nle
  MOD_RES 18: Nle
AVSEIQFXHNLGKHLSSXERVEWLRKKLQDVHNY

SEQ ID NO: 42 - protein, 34 aa, Homo sapiens
  MOD_RES 8: Nle
  MOD_RES 21: Nle
AVSEIQLXHNLGKHLASVERXQWLRKKLQDVHNY

SEQ ID NO: 43 - protein, 34 aa, Homo sapiens
AVSEIQFMHNLGKHLSSMERVEWLRKKLQDVHNY

SEQ ID NO: 44 - protein, 34 aa, Homo sapiens; identical to SEQ ID NO: 41
  MOD_RES 8: Nle
  MOD_RES 18: Nle

SEQ ID NO: 45 - protein, 34 aa, Homo sapiens
  MOD_RES 8: Nle
  MOD_RES 18: Nle
SVSEIQLXHNLGKHLNSXERVEWLRKKLQDVHNY

SEQ ID NO: 46 - protein, 34 aa, Homo sapiens
  MOD_RES 21: Nle
AVSEIQLMHNLGKHLASVERXQWLRKKLQDVHNF

SEQ ID NO: 47 - protein, 34 aa, Homo sapiens
AVSEHQLLHDKGKSIQDLRRRFFLHHLIAEIHTY

SEQ ID NO: 48 - protein, 36 aa, Homo sapiens
AVSEHQLLHDKGKSIQDLRRRIFLQNLIEGVNTAEY